(19) United States
(12) Patent Application Publication
(10) Pub. No.: US 2017/0352289 A1
(43) Pub. Date: Dec. 7, 2017

(54) SYSTEMS AND METHODS FOR REAL-TIME NOTIFICATION AND MOBILE CONTENT DELIVERY

(71) Applicant: Renaissance Periodization, LLC, Charlotte, NC (US)

(72) Inventors: Michael Israetel, Philadelphia, PA (US); Jennifer Case, Kansas City, KS (US); Christian Junge, San Francisco, CA (US)

(21) Appl. No.: 15/614,463

(22) Filed: Jun. 5, 2017

Related U.S. Application Data
(60) Provisional application No. 62/346,396, filed on Jun. 6, 2016.

(51) Int. Cl. G09B 19/00; A61B 5/00; A61B 5/01; A61B 5/021; A63B 24/00; G01G 19/44; H04L 29/06

(52) U.S. Cl. CPC G09B 19/0092; A61B 5/021; A61B 5/4812; A61B 5/486; A61B 5/4866; A63B 24/0062; A63B 24/0075; A61B 5/01; A63B 2024/0078; A63B 2230/015; A63B 2230/062; A63B 2230/305; A63B 2230/505; A63B 2230/705; H04L 63/0272; G01G 19/44

(57) ABSTRACT

Systems and methods for real-time notification and mobile content delivery are provided herein. In one embodiment, a system includes a server that is configured to receive user parameter data of a user, construct a user plan that includes meal and exercise components, perform periodic evaluations of the user based on one or more targets, the one or more targets being based on the meal and exercise components, the evaluations including biometric feedback from biometric tracking devices or the user that are indicative of user performance relative to the one or more targets, and periodically transmit messages to a client device associated with the user to inform the user of any of compliance with or deviations relative to the one or more targets.

### CROSS REFERENCE TO RELATED APPLICATIONS

This application claims benefit and priority of U.S. Provisional Application Ser. No. 62/346,396, filed on Jun. 6, 2016, which is hereby incorporated by reference herein in its entirety including all references and appendices cited therein, for all purposes.

### FIELD OF THE PRESENT DISCLOSURE

The present disclosure relates generally to mobile content delivery and real-time notification, and more specifically, but not by limitation, to systems and methods that provide information to mobile users in a real-time or near real-time manner. An example system can utilize machine learning or other heuristics determine what type of information is delivered and when.

### SUMMARY

Various embodiments of the present disclosure are directed to a system comprising a server that is configured to: receive user parameter data of a user; construct a user plan that comprises meal and exercise components; perform periodic evaluations of the user based on one or more targets that for a time period, the one or more targets being based on the meal and exercise components, the evaluations comprising biometric feedback from biometric tracking devices or the user that are indicative of user performance relative to the one or more targets, regenerate the user plan based on the biometric feedback if the biometric feedback indicates that the user is not achieving the one or more targets within the time period, transmit in real-time or near real-time; and a client device that provides the user parameter data and receives the messages from the server.

Various embodiments of the present disclosure are directed to a method comprising: receiving user parameter data of a user; constructing a user plan that comprises meal and exercise components; performing periodic evaluations of the user based on one or more targets that for a time period, the one or more targets being based on the meal and exercise components, the evaluations comprising biometric feedback from biometric tracking devices or the user that are indicative of user performance relative to the one or more targets; regenerate the user plan based on the biometric feedback if the biometric feedback indicates that the user is not achieving the one or more targets within the time period; and periodically transmit messages to a client device associated with the user to inform the user of any of compliance with or deviations relative to the one or more targets.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1A illustrates an exemplary architecture for practicing aspects of the present technology.

FIG. 1B illustrates another exemplary architecture for practicing aspects of the present technology.

FIGS. 2A-2D collectively illustrate an example method that can be executed in accordance with the present disclosure.

FIG. 3 illustrates an example method for adjusting maintenance of the plan provided to a user.

FIG. 4 illustrates a method for real-time adjusting fat loss criteria in accordance with the present disclosure.

FIG. 5 illustrates an example method for real-time adjusting weight gain for a user.

FIG. 6 is a schematic diagram of an example computer system that can be used to practice aspects of the present disclosure.

### DESCRIPTION OF EXEMPLARY EMBODIMENTS

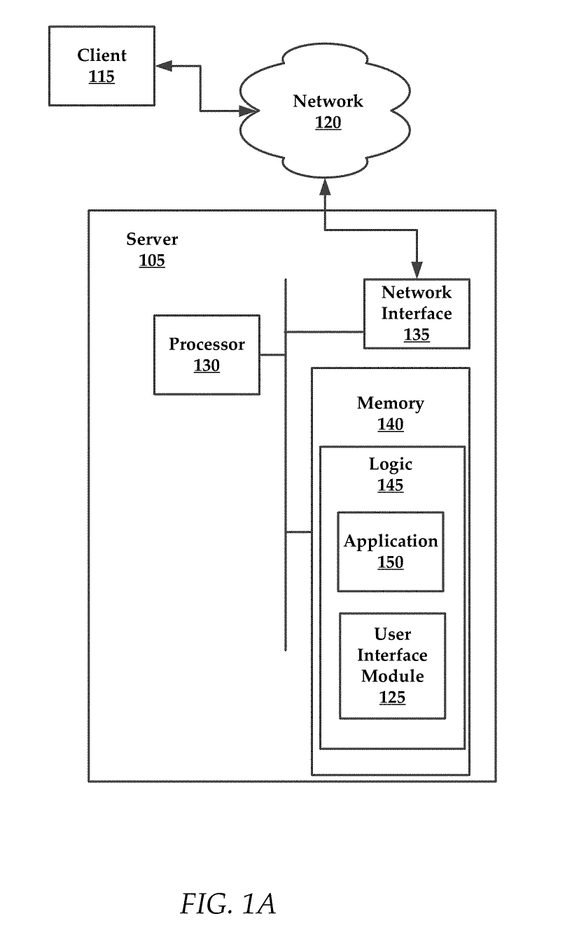

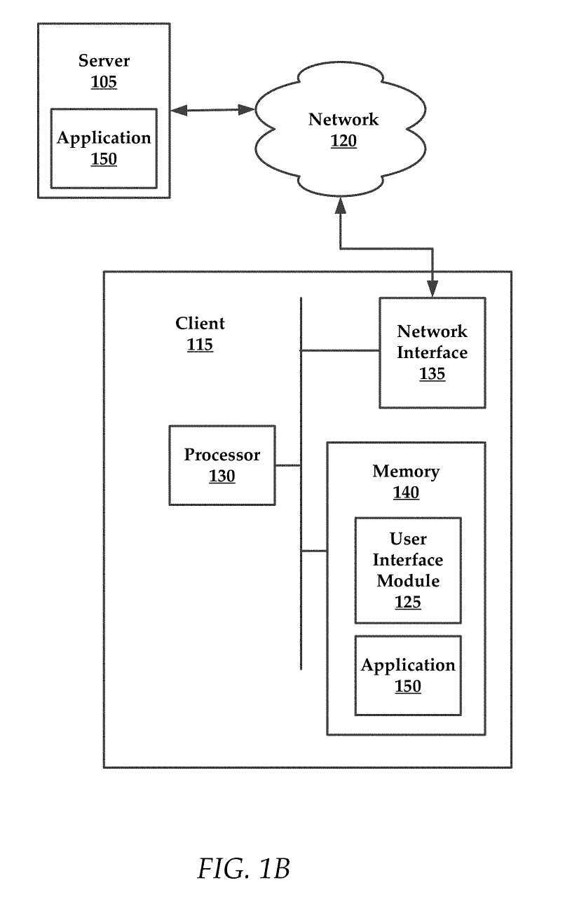

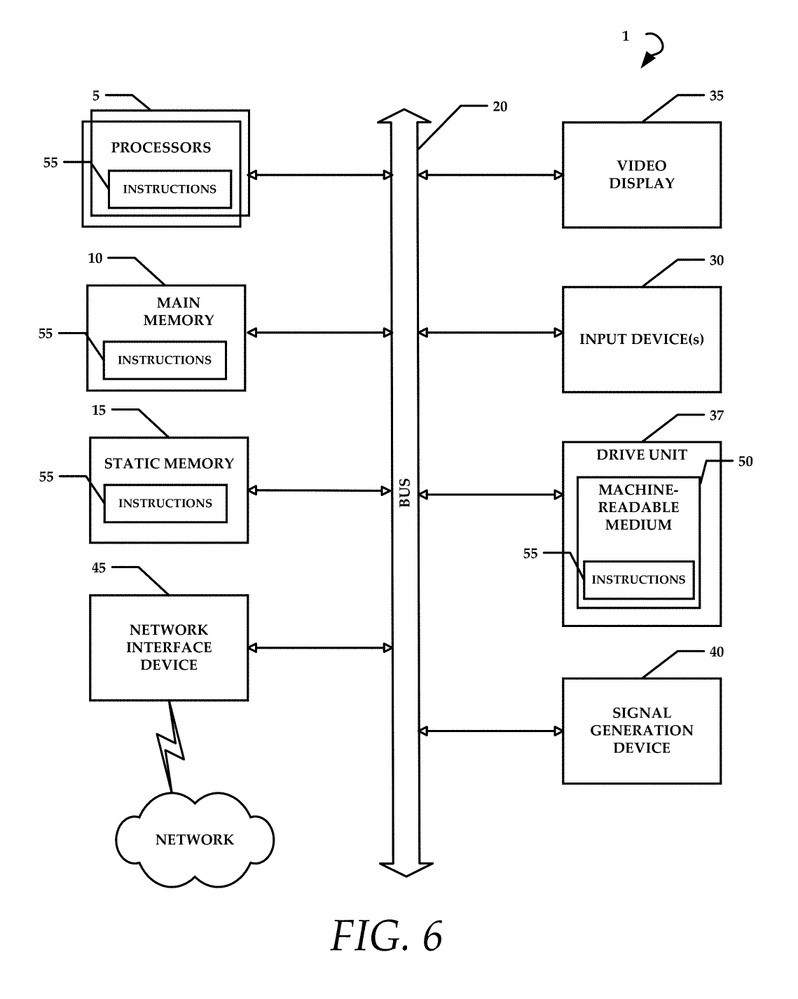

FIG. 1A illustrates an exemplary architecture for practicing aspects of the present disclosure. The architecture comprises a server system, hereinafter “system 105” that is configured to provide various functionalities, which are described in greater detail throughout this document. Generally the system 105 is configured to communicate with client devices, such as client 115. The client 115 may include, for example, a Smartphone, a laptop, a computer, or other similar computing device. An example of a computing device that can be utilized in accordance with the present disclosure is described in greater detail with respect to FIG. 6.

The system 105 may communicatively couple with the client 115 via a public or private network, such as network 120. Suitable networks may include or interface with any one or more of, for instance, a local intranet, a PAN (Personal Area Network), a LAN (Local Area Network), a WAN (Wide Area Network), a MAN (Metropolitan Area Network), a virtual private network (VPN), a storage area network (SAN), a frame relay connection, an Advanced Intelligent Network (AIN) connection, a synchronous optical network (SONET) connection, a digital T1, T3, E1 or E3 line, Digital Data Service (DDS) connection, DSL (Digital Subscriber Line) connection, an Ethernet connection, an ISDN (Integrated Services Digital Network) line, a dial-up port such as a V.90, V.34 or V.34bis analog modem connection, a cable modem, an ATM (Asynchronous Transfer Mode) connection, or an FDDI (Fiber Distributed Data Interface) or CDDI (Copper Distributed Data Interface) connection. Furthermore, communications may also include links to any of a variety of wireless networks, including WAP (Wireless Application Protocol), GPRS (General Packet Radio Service), GSM (Global System for Mobile Communication), CDMA (Code Division Multiple Access) or TDMA (Time Division Multiple Access), cellular phone networks, GPS (Global Positioning System), CDPD (cellular digital packet data), RIM (Research in Motion, Limited) duplex paging network, Bluetooth radio, or an IEEE 802.11-based radio frequency network. The network 120 can further include or interface with any one or more of an RS-232 serial connection, an IEEE-1394 (Firewire) connection, a Fiber Channel connection, an IrDA (infrared) port, a SCSI (Small Computer Systems Interface) connection, a USB (Universal Serial Bus) connection or other wired or wireless, digital or analog interface or connection, mesh or Digi® networking.

The system 105 generally comprises a processor, 130, a network interface 135, and a memory 140. According to some embodiments, the memory 140 comprises logic (e.g., instructions) 145 that can be executed by the processor 130 to perform various methods. For example, the logic may include a user interface module 125 as well as a content management application 150, which are configured to provide the functionalities described in greater detail herein.

It will be understood that the functionalities described herein, which are attributed to the system 105 and application 150 may also be executed within the client 115. That is, the client 115 may be programmed to execute the functionalities described herein. In other instances, the system 105 and client 115 may cooperate to provide the functionalities described herein, such that the client 115 is provided with a client-side application that interacts with the system 105 such that the system 105 and client 115 operate in a client/server relationship. Complex computational features may be executed by the server 105, while simple operations that require fewer computational resources may be executed by the client 115, such as data gathering and data display.

In general, the user interface module 125 may be executed by the system 105 to provide various graphical user interfaces (GUIs) that allow users to interact with the system 105. In some instances, GUIs are generated by execution of the applications 150/155. Users may interact with the system 105 using, for example, a client 115. The system 105 may generate web-based interfaces for the client.

The application 150 can be executed locally on a user device (e.g., client 115), such as a Smartphone or tablet device. Alternatively, the application can be accessed by a user device over a network. Thus, the application 150 can be executed on a server and accessed by the user device using a browser application. The server will serve GUIs of the application 150 as web pages of a standard or mobile website.

In some embodiments, the application 150 is configured to provide the user with a content managed experience related to both inputting of health related parameters, the selection of content management options (e.g., what mode of operation is used such as cutting, maintenance, and/or gain), as well as performance tracking of biometric information, and scheduling and messaging provided to the client 115.

In other embodiments (see FIG. 1B), the UI module 125 and application 150 can reside and execute on the client 115. These embodiments allow for fast and latency reduced performance of both the UI module 125 and the application 150 due to lack of dependency on the network which delivers content from the system 105 to the client 115. The application 150 can reside on the system 105 and prompt interjection of offers into the gameplay provided by the application 150 on an as-needed basis. Additional components of the client 115 are described in greater detail above with reference to FIG. 1A, as part of the system 105. Although not shown, the system 105 would also include any required components for computing, storage, and networking.

In general, the application 150 can be configured to receive a plurality of input parameters for a user such as body composition (weight, height, age, sex), activity levels, job type, and other indicative information. The user can also select a body type image from a plurality of body type images that represent general body image types such as hourglass, apple, pear, and so forth.

The application 150 utilizes this information to calculate BMI body mass index, daily caloric intake, and so forth. The application 150 can utilize more sophisticated calculations in some instances, when more detailed biometric information is available. Example calculations can include base metabolic rate, for example.

The user can then select a goal such as weight loss, weight gain, and weight maintenance—just to name a few. Based on the selected goal, the application 150 implements a plan for the user, referred to above as content management options. The application 150 can determine an ideal workout schedule and meal plan for the user using the received parameters described above.

In some embodiments, the application 150 can leverage a database of workout information and food information to create the workout schedules and meal plans for the user, based on their goals/targets and parameters.

For example, using a current weight and other parameters, the application 150 can determine that the user needs to lose at least 10% of their body fat, relative to established health guidelines. The application 150 can create a meal plan from the food information that includes a plurality of meals for each day which are balanced from a macronutrient standpoint. For example, each meal can have a ratio of protein, fats, and carbohydrates that are tailored to the goal for the individual.

Also, knowing the goal of the individual, the application 150 can select appropriate times during the day for the user to eat their meals. The application 150 can generate and transmit to the client 115 scheduled messages that include prompts for meal time. For example, the application 150 can generate a message that informs the user that breakfast is approaching. The message also includes the exact contents of the meal for ease of use.

In some embodiments, the user can track their food consumption using the application 150. As the user tracks their meal consumption they can also input their weight on a periodic basis. Using the empirical feedback, the application 150 can selectively adjust the workout schedule of the user and/or the meal plans if the goals are not being met. For example, if the user is not losing weight (goal) according to the data entered, the application 150 can selectively adjust the meal plans, for example, by dropping calories or reconfiguring meals (e.g., remove carbohydrates) in order to assist with weight loss. Similar adjustments can be made during other processes such as weight gain and/or weight maintenance. Thus, the system utilizes periodic progress information provided by the client to track the progress of the user.

To be sure, the application 150 is learning from user behaviors and input, measuring the received information against goals, and transmit corrective messages to the user on an as-needed basis when meals are upcoming, meal plans have changed, workout time are scheduled, and other such event or schedule-based messaging are beneficial.

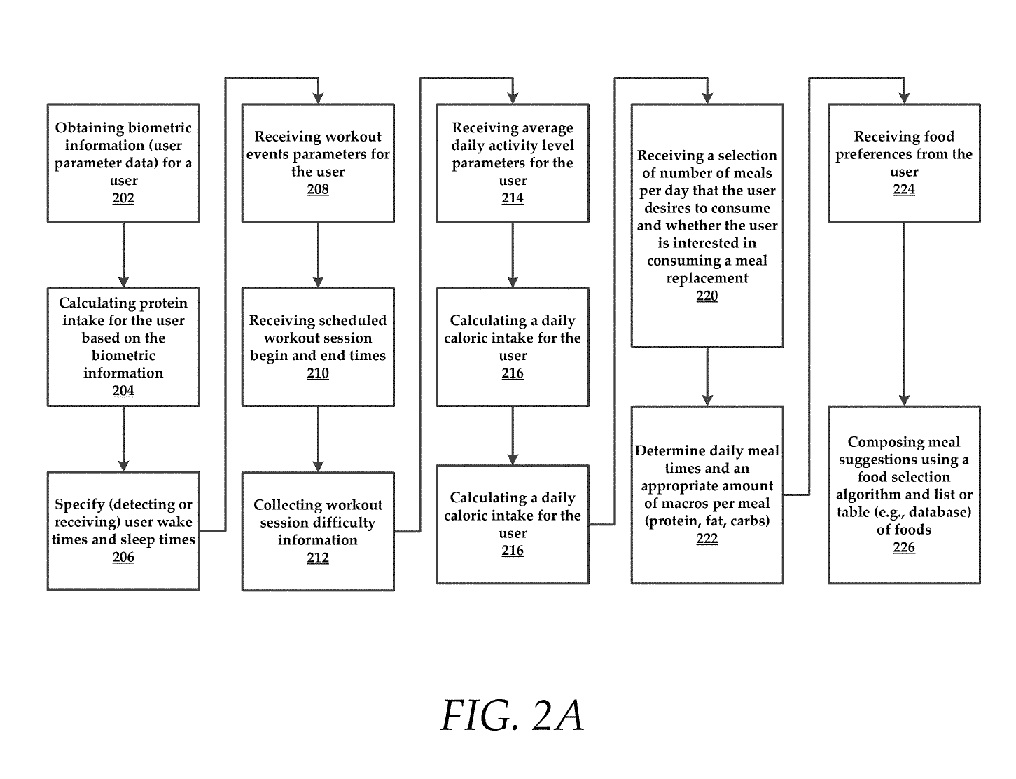

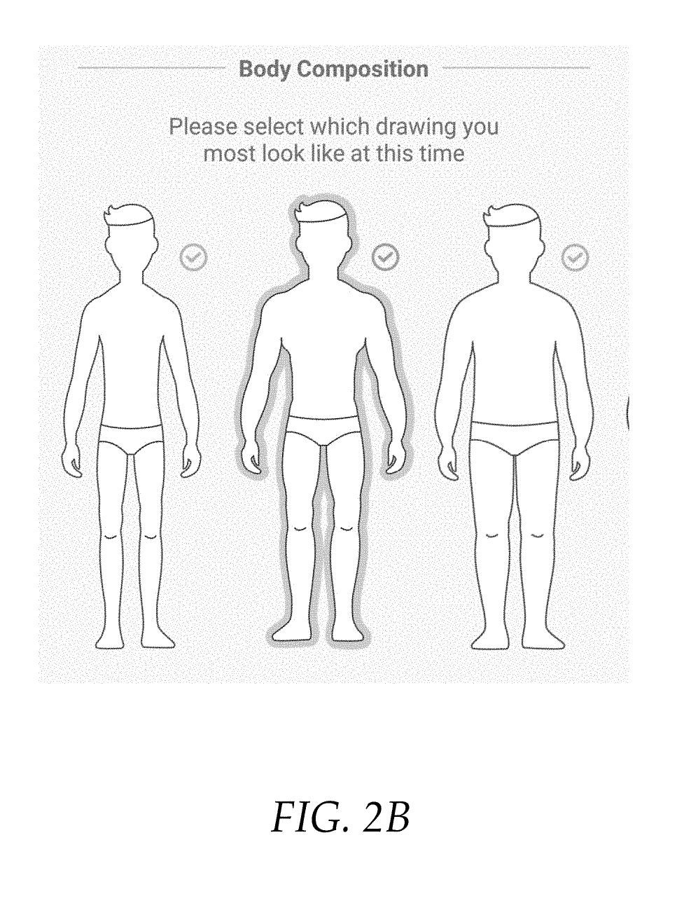

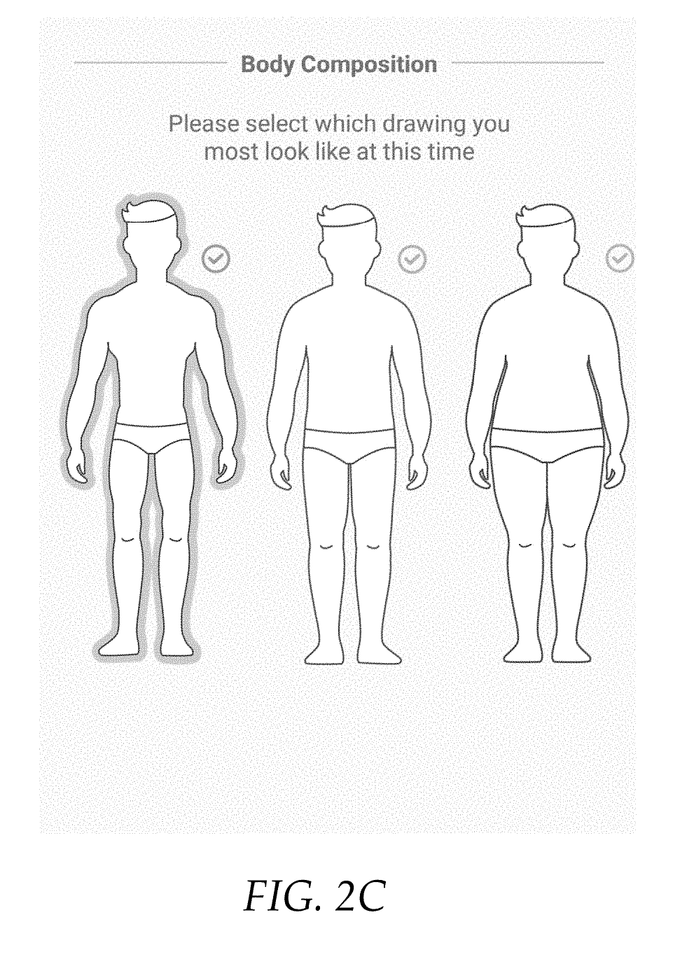

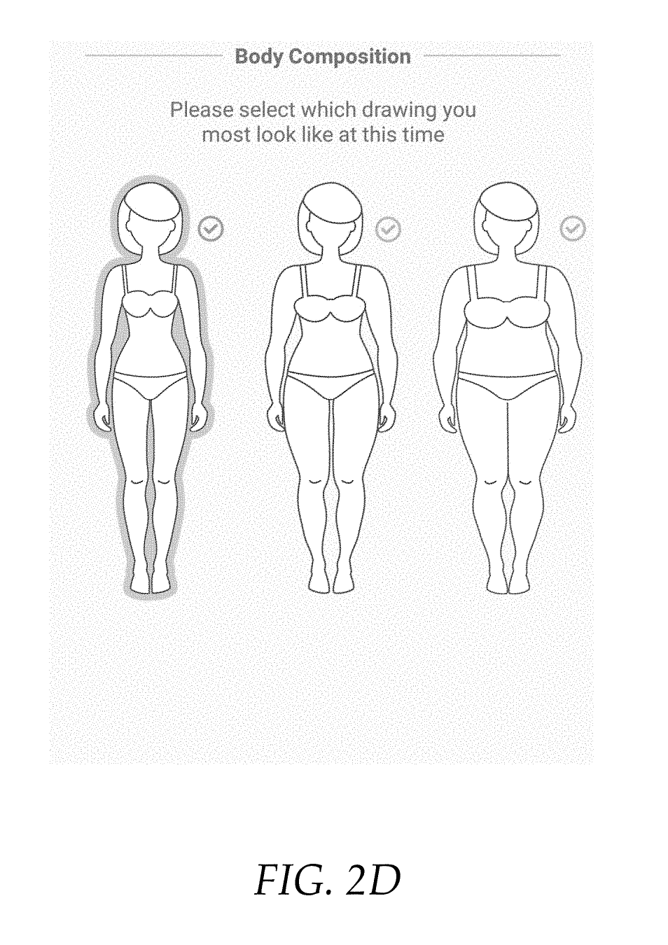

FIGS. 2A-2D collectively illustrate an example method that can be executed in accordance with the present disclosure. The method can be executed using the application 150 of FIGS. 1A and/or 1B. The method includes a step 202 of obtaining biometric information (user parameter data) for a user. This can include, for example, the application requesting a current body weight, an age of the user, a height of the user, a sex of the user, and current body composition. In some embodiments, the selection of current body composition can include the user selecting an image of a body type, such as lean, pear-shaped, hourglass, and so forth. That is, the application displays a plurality of body composition types and the user can select the body type that most closely corresponds to their current body type. These example body types are illustrated in FIGS. 2B-2D.

Next, the method includes a step 204 of calculating protein intake for the user based on the biometric information. In some embodiments the protein intake is a factor of user sex and body composition type. For example, the calculation of protein includes multiplying body weight by a protein weighting coefficient that is based on user sex and body composition type. In one embodiment, if the user is a male that has an athletic body type, the protein weighting coefficient is one. When the user is a female with a pear-shaped body type the protein weighting coefficient is 0.8.

In another step 206, the user can specify their wake times and sleep times. This information can be obtained from a sleep tracking biometric device. In other instances this information can be entered by a user through a graphical user interface provided by the application.

The method also includes a step 208 of receiving workout event parameters for the user. This information includes a current number of days in the week when the user is exercising.

In one or more embodiments, the method includes a step 210 of receiving workout session begin and end times. As with the steps above, this method can include manual input from the user or automated calculation based on a biometric device used by the user, such as a heart rate tracking wearable other similar device.

In some embodiments, the method includes a step 212 of collecting workout session difficulty information. Again, this can include information obtained from a heart rate tracking wearable worn during a workout session, or from manual user input. This can include how often during the week or in a day that the user is training.

According to the present disclosure, the method also includes a step 214 of receiving average daily activity level parameters, which correspond to an activity level of the user outside of the workout information specified above. In some embodiments the application can receive as input an option to vary or create alternative scheduled workouts for the user.

In each of steps 206-214, the application can provide the user with selectable options that correspond to input for each of the facets described above. For example, with respect to a daily activity level, the application can provide the user with selections such as (a) Sedentary: I spend most of my day doing tasks while seated, and I rarely get out of breath or feel physically pushed outside of training; (b) Mildly Active: I spend most of my day doing tasks while seated, but I'm on my feet a bit too and get to move around some; (c) Moderately Active: I spend a good deal of my day on my feet and moving, and sometimes I get pretty physically tired from my daily tasks; and (d) Heavily Active: I spend most of my day on my feet, moving around and doing pretty hard physical work. I'm often pushed pretty hard physically during my daily tasks.

These are merely examples of selectable options. Additional or fewer options may be presented to the user. Notwithstanding, each of the selections is associated with measurable value that is used to calculate, for example, caloric intake for the user.

Thus, in some embodiments, using the information obtained in steps 206-214, the application can then provide a step 216 of calculating a daily caloric intake for the user. In some embodiments, the application can use a Harris-Benedict equation. These values can be modified and/or tailored using a multiplication factor that is based on activity level, training information, and carbohydrate intake—just to name a few.

The method can also comprise a step of calculating additional macronutrient ratio values, such as carbohydrate intake and fat intake in step 218.

In some embodiments, the method comprises a step 220 of receiving a selection of number of meals per day that the user desires to consume and whether the user is interested in consuming a meal replacement. Using this information the application is configured to determine daily meal times and an appropriate amount of macros per meal (protein, fat, carbs) in step 222. This can include applying a ruleset that specifies meal times based on previous meal times, workout sessions, and so forth. In some embodiments, remaining (not first, postworkout or last meals) are placed with the following rule structure (this is a priority-rule structure, so the first rule should be attempted to be filled first, then the second, then the third, then the fourth: (1) equidistantly between post-workout meal and bedtime meal unless five hours or less exists between them; (2) equidistantly between the waking meal and workout one unless less than four hours or less exists between waking meal and first workout; (3) equidistantly between first post-workout meal and second workout unless four hours or less exists between post-workout meal for the first workout and the beginning of the second workout; and (4) in any other interval where greater than six hours passes between meals. The meal should be placed equidistantly between the previous meal and the next meal.

Protein per meal can be calculated by multiplying total daily protein by hours in meal interval until next meal divided by 24 hours. The same equation can be used to calculate carbohydrates per meal, as well as fat per meal. The ratio of the macronutrients and amounts consumed depend on if the user is training/working out or not.

In step 224, the method can comprise receiving food preferences from the user. Using these guidelines the method includes a step 226 of composing meal suggestions using a food selection algorithm and list or table (e.g., database) of foods (where foods are defined by macronutrient components per gram) that includes (a) for all non-shake meals, the app allots the following first grams of carbs in each meal to be consumed through the “veggies” column: ((pounds bodyweight of individual)/15)); (b) For remaining post-veggie carbs, calculate the amount of each carb source (in raw weight) it would take to fill in each meal's carb allotment; (c) calculate protein serving sizes (total raw weight) from the protein list. This yields the final per-meal protein serving amount; (d) calculate fat amount serving sizes (total raw weight) from a list of healthy fats; (e) take ancillary carb amounts from steps c, d subtract them from the outcome of step b. This is the final per-meal carb amount; and then (f) obtain ancillary fat amounts from step c and e and subtract that from the output of step d. This is the final per-meal healthy fat serving amount.

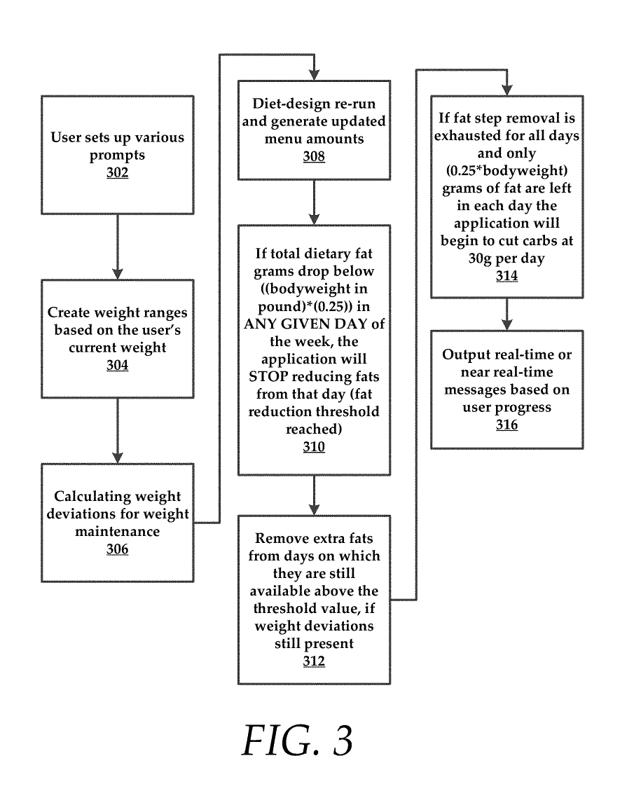

FIG. 3 illustrates an example method for adjusting maintenance of the plan provided to a user. In some embodiments, if a specified period of time, such as a week, has elapsed since the user utilized the application to perform the method of FIGS. 2A-2D, the user can be prompted to re-input updated data. The user sets up various prompts in step 302, such as how often they will enter their body weight, as well as the days on which the body weight will be obtained. The user can also setup notifications or prompts to remind the user to enter their weight in step 304. In some embodiments, this information can be obtained from a wearable or a wireless enabled scale.

The application can then create weight ranges based on the user's current weight in step 304. For example, the application can create a range that is +/−2.5% of current body weight. The method includes a step of calculating weight deviations in step 306. For example, after one calendar week of weigh-ins (on the morning of the 6th day, day before the week repeats), the application will calculate the AVERAGE deviation from the original phase-starting weight. If average is within 2.5% of initial weight, the method does not alter the diet. If average is lower than 2.5%, then the method includes outputting suggestions that result in food options where an increase total daily fats by 30 g and diet-design is re-run and generate updated menu amounts. This process is repeated for every week during which average is below 2.5% margin.

If average is lower than 2.5%, then the application will output suggestions that result in food options where a decrease total daily fats by 30 g and diet-design re-run and generate updated menu amounts occurs in step 308. Next, the process is repeated for every week during which average is below 2.5% margin.

If total dietary fat grams drop below ((bodyweight in pound)*(0.25)) in ANY GIVEN DAY of the week, the application will STOP reducing fats from that day (fat reduction threshold reached), in step 310. Also, for any day(s) of the week, the application will remove extra fats from days on which they are still available above the threshold value of (0.25 bodyweight). 30 g multiplied by seven days, in step 312. This means a total of 210 g of fat per week reduction is suggested by the application. Next, if the fat step removal is exhausted for all days and only (0.25*bodyweight) grams of fat are left in each day the application will begin to cut carbs at 30 g per day (with no bottom limit), in step 314. If certain food choices (fattier meats) don't fit into the diet even with no added fat sources (healthy fats), the application will de-limit those options from selection.

In essence, the fat components of the user's meals are iteratively reduced until reaching the fat reduction threshold. After that, the carb components of the user's meals are iteratively reduced along until deviations in the user's weight are brought in range of a desired weight. For example, if the deviations include no more than +5 pounds, when the user's weight is more than five pounds, the aforementioned fat and carb readjustments occur until the user's weight is within the allowable range of deviations.

In some embodiments, the application can output real-time or near real-time messages in step 316. For example, if an updated diet from is unchanged, the application will prompt user with “You're on track! Please continue on your current diet for the next week.” If the updated diet includes different fat or carb components the application will prompt the user with “Based on your progress, your diet has been adjusted for this next week.” Specific dietary changes are displayed to the user, which include a link to the updated diet for the following time period, such as a week.

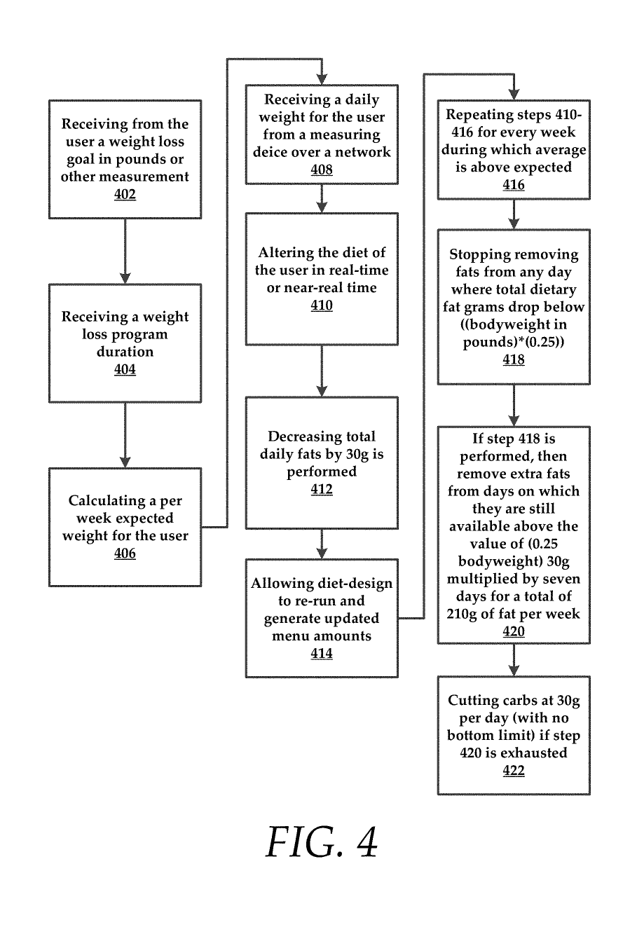

FIG. 4 illustrates a method for real-time adjusting fat loss criteria in accordance with the present disclosure. Again, this adjustment threshold will depend on the current weight, height, sex, activity levels, and so forth determined for the user. The method includes a step 402 of receiving from the user a weight loss goal in pounds or other measurement. The allowable weight loss goal is adjusted based on health criteria, such as a safe weekly weight loss threshold. Delimiters such as fat percentages, both lower and upper can be used. Another delimiter can include now allowing a user to go below a BMI of 17.

In another step 404, the method includes receiving a weight loss program duration. Again, these selections are available when compliant with the safe weekly/daily weight loss threshold. In some embodiments, a minimum rate of loss is 0.25% current body mass per week and a maximum rate of loss is 1% current body mass per week.

The application then calculates a per week expected weight for the user in step 406. This value corresponds to, and is calculated using the values in step 402 and 404.

In some embodiments, the method includes a step 408 of receiving a daily weight for the user, for example from a selectable menu or from output of a device, such as a wireless or smart scale that is coupled with a server over a network connection.

In various embodiments, after a period of time, such as a calendar week, the method can include altering the diet of the user in real-time or near-real time in step 410. For example, after one calendar week of weigh-ins (on the morning of the 6th day, day before the week repeats), the application can compare an entered weight weekly AVERAGE for that last week to the expected weight established in step 406. If the average is higher than expected, changes occur.

In an example sub-method for adjusting the user's diet, a step 412 of decreasing total daily fats by 30 g is performed. This adjustment threshold will depend on the current weight, height, sex, activity levels, and so forth determined for the user.

Next, a step 414 of allowing diet-design to re-run and generate updated menu amounts. In another embodiment, a step 416 includes repeating steps 412-416 for every week during which average is above expected. When total dietary fat grams drop below ((bodyweight in pounds)*(0.25)) in ANY GIVEN DAY of the week, the method includes stopping removing fats from that day in step 418.

If step 418 occurs for any day(s) of the week, the method includes removing extra fats from days on which they are still available above the value of (0.25 bodyweight) 30 g multiplied by seven days for a total of 210 g of fat per week to remove, in step 420.

After step 420 has been exhausted for all days and only (0.25*bodyweight) grams of fat are left in each day, the method includes step 422 of cutting carbs at 30 g per day (with no bottom limit). If carbs are cut to within one cut of zero (30 g per day on all days of the week), inform the user “The strictness of your diet has entered a level in which further calorie reductions cannot be guaranteed as safe. The diet will now return you to maintenance at this current weight, and we would like to you reach out to a coach for further attempts at dieting if you're interested.” This message includes a link to a weight loss professional.

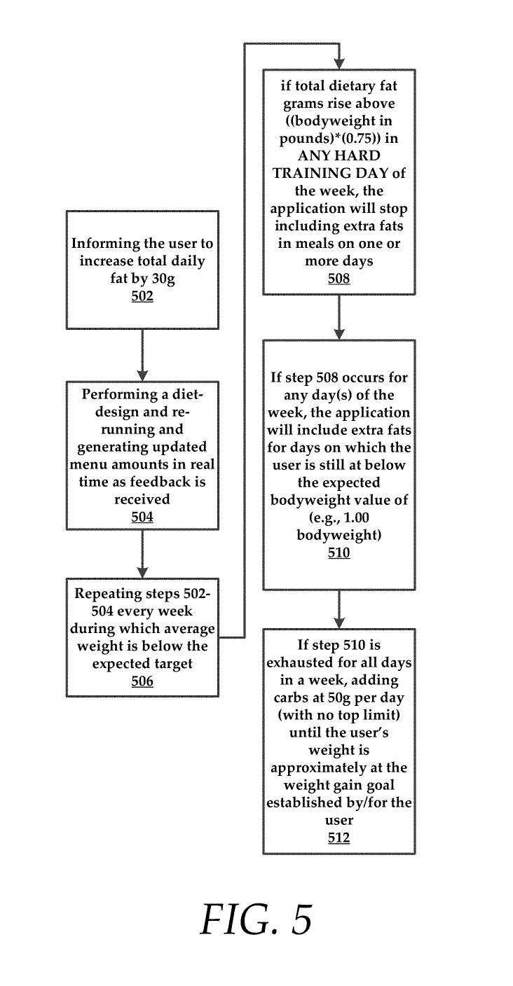

FIG. 5 illustrates an example method for real-time adjusting weight gain for a user. The method is similar to that of adjusting weight loss but the algorithms applied vary substantially. While questions related to how much and how quickly the user wishes to gain weight, the method will calculate metrics for weight gain related to muscle growth rather than fat loss.

In some embodiments, the method includes a step 502 of informing the user to increase total daily fat by 30 g. Again, this adjustment threshold will depend on the current weight, height, sex, activity levels, and so forth determined for the user.

In one embodiment, the method includes a step 504 of performing a diet-design and re-running and generating updated menu amounts. This can occur as real-time feedback/metrics are received from the user or from one or more biometric sensing devices. Next, the method includes a step 506 of repeating steps 502 and 504 every week during which average weight is below expected weight. According to some embodiments, the method includes a step 508 where if total dietary fat grams rise above ((bodyweight in pounds)*(0.75)) in ANY HARD TRAINING DAY of the week, the application will stop including extra fats in meals on one or more days.

In some embodiments, if step 508 occurs for any day(s) of the week, the application will include extra fats for days on which the user is still at below the expected bodyweight value of (e.g., 1.00 bodyweight). In one embodiment, 30 g multiplied by seven days results in a total of 210 g of fat per week which is added. When step 510 has been exhausted for all days and all days have reach their top-end fat limits (e.g., fat increase threshold), the method includes a step 512 of adding carbs at 50 g per day (with no top limit) until the user's weight is approximately at the weight gain goal established by/for the user.

FIG. 6 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a personal digital assistant (PDA), a cellular telephone, a portable music player (e.g., a portable hard drive audio device such as an Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The disk drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a network (e.g., network 120) via the network interface device 45 utilizing any one of a number of well-known transfer protocols (e.g., Hyper Text Transfer Protocol (HTTP)). While the machine-readable medium 50 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

One skilled in the art will recognize that the Internet service may be configured to provide Internet access to one or more computing devices that are coupled to the Internet service, and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the present disclosure in the form disclosed. Many modifications and variations will be apparent to those of ordinary skill in the art without departing from the scope and spirit of the present disclosure. Exemplary embodiments were chosen and described in order to best explain the principles of the present disclosure and its practical application, and to enable others of ordinary skill in the art to understand the present disclosure for various embodiments with various modifications as are suited to the particular use contemplated.

Aspects of the present disclosure are described above with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated.

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present disclosure. As such, some of the components may have been distorted from their actual scale for pictorial clarity.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In the following description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It is noted at the outset that the terms “coupled,” “connected”, “connecting,” “electrically connected,” etc., are used interchangeably herein to generally refer to the condition of being electrically/electronically connected. Similarly, a first entity is considered to be in “communication” with a second entity (or entities) when the first entity electrically sends and/or receives (whether through wireline or wireless means) information signals (whether containing data information or non-data/control information) to the second entity regardless of the type (analog or digital) of those signals. It is further noted that various figures (including component diagrams) shown and discussed herein are for illustrative purpose only, and are not drawn to scale.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel, or may be performed at different times.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. A system comprising:
a server that is configured to:
receive user parameter data of a user;
construct a user plan that comprises meal and exercise components;
perform periodic evaluations of the user based on one or more targets that for a time period, the one or more targets being based on the meal and exercise components, the evaluations comprising biometric feedback from biometric tracking devices or the user that are indicative of user performance relative to the one or more targets;
regenerate the user plan based on the biometric feedback if the biometric feedback indicates that the user is not achieving the one or more targets within the time period; and
transmit in real-time or near real-time messages to a client device associated with the user to inform the user of any of compliance with or deviations relative to the one or more targets; and
a client device that provides the user parameter data and receives the messages from the server.

2. The system according to claim 1, wherein the meal information and exercise information that are driven by scheduling.

3. The system according to claim 1, wherein the biometric feedback comprises a weight of the user received from a wireless or smart scale.

4. The system according to claim 1, wherein the biometric feedback comprises any of a heart rate, a blood pressure, a temperature, wake and sleep cycles of the user received from a wearable device configured to measure and transmit the biometric feedback.

5. The system according to claim 1, wherein the user parameter data comprises a selection of a body composition type.

6. The system according to claim 1, wherein the server is further configured to:
display a plurality of body composition type representations; and
receive a body composition type selection of the plurality of body composition type representations from the client device.

7. The system according to claim 1, wherein the server is further configured to:
calculate deviations in a weight of the user over time that are indicative of weight gain relative to an increase in body fat percentage;
iteratively adjusting a fat component of the meal component of the user plan until the deviations are within a desired range;
regenerating the user plan to update meal suggestions in accordance with the iterative adjustment in the fat component;
wherein if the fat component is reduced to a fat reduction threshold, the method further comprises iteratively adjusting a carbohydrate component of the meal component of the user plan until the deviations are within the desired range; and
regenerating the user plan to update meal suggestions in accordance with the iterative adjustment in the carbohydrate component.

8. The system according to claim 7, wherein the server is further configured to apply a delimiter that prevents the user from selecting a weight loss goal of the one or more goals where the user's body mass index (BMI) would be reduced below 17.

9. The system according to claim 7, wherein the server is further configured to apply a delimiter that prevents the user from selecting a weight loss goal of the one or more goals where the user's weight loss would be reduced below more than two pounds per week.

10. The system according to claim 7, wherein the server is further configured to apply a delimiter that prevents a reduction in the fat component that would result in a total fat component of the meal plan dropping an amount that is greater than the user's body weight multiplied by approximately 0.25.

11. The system according to claim 1, wherein the server is further configured to:
establish weight gain criteria comprising a target weight that is to be achieved over a period of time;
iteratively adjusting a fat component of the meal component of the user plan so that the user's weight is increasing in accordance with the weight gain criteria over the period of time;
regenerating the user plan to update meal suggestions in accordance with the iterative adjustment in the fat component;
wherein if the fat component is increased to a fat increase threshold, the method further comprises iteratively adjusting a carbohydrate component of the meal component of the user plan until the user's weight has increased to the target weight; and
regenerating the user plan to update meal suggestions in accordance with the iterative adjustment in the carbohydrate component.

12. The system according to claim 1, wherein the meal component comprises a total caloric intake that is adjusted using a weighting factor based on any of activity level, training information, and carbohydrate intake.

13. The system according to claim 1, wherein the meal component of the user plan comprises selected times for the user for consuming meals.

14. The system according to claim 13, wherein the selected times are based on wake times, workout times, and last consumed meal.

15. A method, comprising:
receiving user parameter data of a user;
constructing a user plan that comprises meal and exercise components;
performing periodic evaluations of the user based on one or more targets, the one or more targets being based on the meal and exercise components, the evaluations comprising biometric feedback from biometric tracking devices or the user that are indicative of user performance relative to the one or more targets; and
periodically transmitting messages to a client device associated with the user to inform the user of any of compliance with or deviations relative to the one or more targets.

16. The method according to claim 16, further comprising constructing a selected times for the user to consume a meal such that:
the meal is equidistantly between a post-workout meal and bedtime meal unless five hours or less exists between them;
the meal is equidistantly between the waking meal and workout one unless less than four hours or less exists between a waking meal and a first workout;
the meal is equidistantly between first post-workout meal and second workout unless four hours or less exists between post-workout meal for the first workout and a beginning of the second workout; and
wherein in any other interval where greater than six hours passes between meals, the meal should be placed equidistantly between a previous meal and a next meal.
